(19) United States
(12) Patent Application Publication
(10) Pub. No.: US 2026/0117162 A1
(43) Pub. Date: Apr. 30, 2026

(54) FOOD POISONING BACTERIA DETECTION SENSOR

(71) Applicant: POSTECH Research and Business Development Foundation, Pohang-si (KR)

(72) Inventors: Youngki KIM, Pohang-si (KR); Yena CHOI, Pohang-si (KR); Sangmin JEON, Pohang-si (KR); Hyunsoo HAN, Pohang-si (KR)

(73) Assignee: POSTECH Research and Business Development Foundation, Pohang-si (KR)

(21) Appl. No.: 19/343,198

(22) Filed: Sep. 29, 2025

(30) Foreign Application Priority Data
Oct. 31, 2024 (KR) 10-2024-0151837

(51) Int. Cl. C12M 1/34; C12M 1/00; C12Q 1/04

(52) U.S. Cl. CPC C12M 41/36; C12M 23/34; C12Q 1/04

(57) ABSTRACT

The present disclosure relates to a food poisoning bacteria detection sensor. According to an embodiment, it is possible to provide the food poisoning bacteria detection sensor including: a substrate part; a liquid crystal part that is formed on the substrate part and is composed of a micro partition wall partitioning a unit pixel and a liquid crystal structure layer disposed inside the partitioned unit pixel; and an accommodating part that accommodates the substrate part and the liquid crystal part and includes an aqueous solution.

### CROSS-REFERENCE TO RELATED PATENT APPLICATION

This application claims the benefit of Korean Patent Application No. 10-2024-0151837, filed on Oct. 31, 2024, in the Korean Intellectual Property Office, the disclosure of which is incorporated herein in its entirety by reference.

### TECHNICAL FIELD

The present disclosure relates to a food poisoning bacteria detection sensor. The present disclosure was derived from research conducted with support from Samsung Electronics.

### BACKGROUND

According to reports of the Ministry of Food and Drug Safety of the Republic of Korea and the U.S. Centers for Disease Control and Prevention (CDC), hundreds to thousands of food poisoning incidents are reported worldwide every year.

Food poisoning occurs through consumption of contaminated food and causes symptoms such as vomiting, abdominal pain, diarrhea, and fever, and thus threatens food stability and safety of people's dietary lives. In particular, as demands for delivery food and convenient food have exploded due to a recent COVID-19 pandemic, social interest in food safety is increasing.

An example of a conventional food poisoning bacteria detection method is a polymerase chain reaction (PCR), and the PCR is a method of amplifying a part of a small amount of DNA strand and is used to enrich a trace amount of food poisoning bacteria present in food. A main process of the PCR consists of a denaturing step, an annealing step, and an extending step, and there are limitations in that specialized equipment for repeatedly operating dozens of cycles over a wide temperature range is required, a process thereof is complex, and a significant amount of time is required for a heating and cooling process.

More specifically, the PCR has limitations of 1) a complex detection process and 2) difficult immediate detection, and when reviewing the complex detection process, a process of enriching the trace amount of food poisoning bacteria and then separating, amplifying, testing, and confirming the same is required, and thus the complex detection process has a limitation of requiring specialized manpower and equipment.

In addition, reviewing the difficult immediate detection, the conventional food poisoning bacteria detection method has limitations in that more than 12 hours for enrichment is required (e.g., heating and cooling process, etc.) and an additional process (e.g., DNA separation, amplification, testing, etc.) for food poisoning bacteria detection is required even after enrichment, and thus immediate detection is difficult.

Accordingly, there is a need for development of a food poisoning bacteria detection sensor that is user-friendly, has an easy pretreatment process, and may immediately detect, which overcomes the limitations of the conventional food poisoning bacteria detection method.

### SUMMARY

### Technical Problem

A food poisoning bacteria detection sensor according to an embodiment of the present disclosure, which is capable of immediate detection as a detection speed thereof is very fast (fast detection speed), is capable of being easily used by non-experts as specialized equipment or specialized technique is not required and a pretreatment process is easy at the same time (user-friendly), is capable of confirming detection of a generated optical signal using a cellphone camera (easy detection), is capable of confirming a quantitative concentration of detected food poisoning bacteria in addition to a presence of the food poisoning bacteria (confirmation of quantitative concentration), and is capable of economical utilization using a liquid crystal, has been proposed to solve the above-described problems.

### Technical Solution

According to an embodiment, it is possible to provide a food poisoning bacteria detection sensor including: a substrate part; a liquid crystal part that is formed on the substrate part and is composed of a micro partition wall partitioning a unit pixel and a liquid crystal structure layer disposed inside the partitioned unit pixel; and an accommodating part that accommodates the substrate part and the liquid crystal part and includes an aqueous solution.

In addition, the liquid crystal structure layer may include a liquid crystal structure that is composed of liquid crystal molecules oriented perpendicular to a substrate-liquid crystal structure layer interface part and an aqueous solution-liquid crystal structure layer interface part, and an organic ionic substance that is disposed at the aqueous solution-liquid crystal structure layer interface part.

In addition, the substrate part may include a glass substrate on which a coating material inducing the liquid crystal molecules to be oriented perpendicular to the substrate-liquid crystal structure layer interface part is coated, and the coating material may be dimethyloctadecyl 3-(trimethoxysilyl)propyl ammonium chloride (DMOAP).

In addition, the liquid crystal molecules may be changed in an orientation in a predetermined direction by at least one of food poisoning bacteria culture media flowing into the accommodating part and a food poisoning bacteria mixture in which food poisoning bacteria and the food poisoning bacteria culture media are mixed, and an optical appearance change of the liquid crystal structure may be induced by the orientation change of the liquid crystal molecules.

In addition, the food poisoning bacteria culture media may include charged side chain amino acid including at least one substance selected from a group consisting of glutamic acid and aspartic acid, and the food poisoning bacteria may be bacteria that include lipopolysaccharide including a positively charged group and carbon chains as a major component of an outer membrane.

In addition, the charged side chain amino acid may induce the orientation change of the liquid crystal molecules by being adsorbed to the aqueous solution-liquid crystal structure layer interface part, and the liquid crystal molecules may be oriented in a direction horizontal to the aqueous solution-liquid crystal structure layer interface part according to a change in an easy axis caused by adsorption of the side chain amino acid to the interface part.

In addition, an adsorption amount of the interface part of the charged side chain amino acid may increase as a pH value of the aqueous solution increases, and the lipopolysaccharide may bind with the charged side chain amino acid to accelerate a speed at which the side chain amino acid is adsorbed to the aqueous solution-liquid crystal structure layer interface part.

In addition, the organic ionic substance may be fixedly disposed by being self-assembled at the aqueous solution-liquid crystal structure layer interface part and may induce the liquid crystal molecules to be oriented perpendicular to the aqueous solution-liquid crystal structure layer interface part.

In addition, the organic ionic substance may include a head group, a carbon chain group, and an anion, the head group may be any one substance selected from a sulfate group and an imidazolium group, a length of the carbon chain group may be 8 to 12, and the anion may be any one substance selected from Br⁻, BF₄⁻, and PF₆⁻.

In addition, when the food poisoning bacteria mixture flows into the accommodating part, a speed at which the liquid crystal molecules are changed in an orientation from vertical to horizontal increases as a concentration of the flown food poisoning bacteria increases, and the optical appearance change of the liquid crystal structure may be accelerated according to the speed increase in the orientation change.

In addition, the speed at which the liquid crystal molecules are changed in the orientation from vertical to horizontal increases as a length of the carbon chain group included in the organic ionic substance decreases, and the optical appearance change of the liquid crystal structure may be accelerated according to the speed increase in the orientation change.

In addition, the liquid crystal structure layer may include a droplet-shaped liquid crystal structure that is composed of liquid crystal molecules oriented in radial configuration and having a topological defect in a center thereof, and an organic ionic substance disposed at an aqueous solution-droplet-shaped liquid crystal structure interface part.

According to an embodiment, it is possible to provide a method of manufacturing a food poisoning bacteria detection sensor including: a first step of preparing a substrate part; a second step of forming a liquid crystal part that is composed of a micro partition wall partitioning a unit pixel and a liquid crystal structure layer disposed inside the partitioned unit pixel on the substrate part; and a third step of disposing the substrate part in an accommodating part that accommodates the liquid crystal part and the substrate part and includes an aqueous solution.

### Advantageous Effects

A food poisoning bacteria detection sensor according to an embodiment of the present disclosure is capable of immediate detection as a detection speed thereof is very fast (fast detection speed), is capable of being easily used by non-experts as specialized equipment or specialized technique is not required and a pretreatment process is easy at the same time (user-friendly), is capable of confirming detection of a generated optical signal using a cellphone camera (easy detection), is capable of confirming a quantitative concentration of detected food poisoning bacteria in addition to a presence of the food poisoning bacteria (confirmation of quantitative concentration), and is capable of economical utilization using a liquid crystal.

### BRIEF DESCRIPTION OF THE DRAWINGS

FIG. 1 is a view for schematically describing a food poisoning bacteria detection sensor according to an embodiment of the present disclosure.

FIG. 2 is a view for describing a food poisoning bacteria detection sensor according to an embodiment of the present disclosure in detail.

FIG. 3 is a view showing an optical signal detection result of a food poisoning bacteria detection sensor according to the present disclosure.

FIG. 4 is a view showing an optical signal detection result according to a pH value of the food poisoning bacteria detection sensor.

FIG. 5 is a view showing a measurement result of a magnitude of optical retardation of the food poisoning bacteria detection sensor according to the present disclosure.

FIG. 6 is a view showing an optical signal detection result according to Salmonella concentration.

FIG. 7 is a view showing an optical signal detection result according to a length of a carbon chain group of an organic ionic substance.

FIG. 8 is a view for describing a food poisoning bacteria detection sensor based on a droplet-shaped liquid crystal structure.

FIG. 9 is a view showing a response time measurement result of the food poisoning bacteria detection sensor based on the droplet-shaped liquid crystal structure.

FIG. 10 is a view for describing a Salmonella selectivity of the food poisoning bacteria detection sensor according to the present disclosure.

FIG. 11 is an evaluation result of interaction energy between bacteria and charged side chain amino acid.

### DETAILED DESCRIPTION OF THE EMBODIMENTS

Preferred embodiments of the present disclosure will be described in detail with reference to the attached drawings in order to fully understand the configuration and effects of the present disclosure.

The present disclosure is not limited to the embodiments disclosed herein, but can be implemented in various forms and be subject to various modifications and changes. However, the present disclosure is provided solely to ensure that the disclosure of the present disclosure is complete through the description of the present embodiments and to fully inform those having ordinary skill in the art of the scope of the disclosure. The components in the attached drawings are enlarged from their actual size for convenience of the description, and the proportions of each component may be exaggerated or reduced.

The terms used herein is for the purpose of describing the embodiments only and is not intended to be limiting of the disclosure. In addition, the terms used herein may be interpreted as having a meaning commonly known to those having ordinary skill in the art unless otherwise defined. In addition, as used herein, the singular forms may include the plural forms unless the context clearly dictates otherwise. The terms “comprises” and/or “comprising” used herein specify the presence of a referenced component, step, operation, and/or element, but do not exclude the presence or addition of one or more other components, steps, operations, and/or elements.

In the present specification, when a layer is referred as being ‘on’ another layer, it may be formed directly on the surface of the other layer, or there may be a third layer interposed therebetween. As used herein, the terms first, second, etc. are used to describe various regions, layers, etc., but these regions and layers should not be limited by these terms. These terms are only used to distinguish one region or layer from another. Thus, a part referred to as a first part in one embodiment may be referred to as a second part in another embodiment. The embodiments described and exemplified herein also include complementary embodiments thereof. Like reference numerals may refer to like or corresponding components throughout the specification.

The present disclosure relates to a food poisoning bacteria detection sensor capable of easy detection of food poisoning bacteria without specialized manpower or specialized equipment by exhibiting an immediate optical signal according to a presence of the food poisoning bacteria.

Specifically, the food poisoning bacteria detection sensor of the present disclosure includes a liquid crystal structure that is composed of liquid crystal molecules and may exhibit an immediate optical signal by using an optical appearance change of the liquid crystal structure according to an orientation change of the liquid crystal molecules.

### Food Poisoning Bacteria Detection Sensor

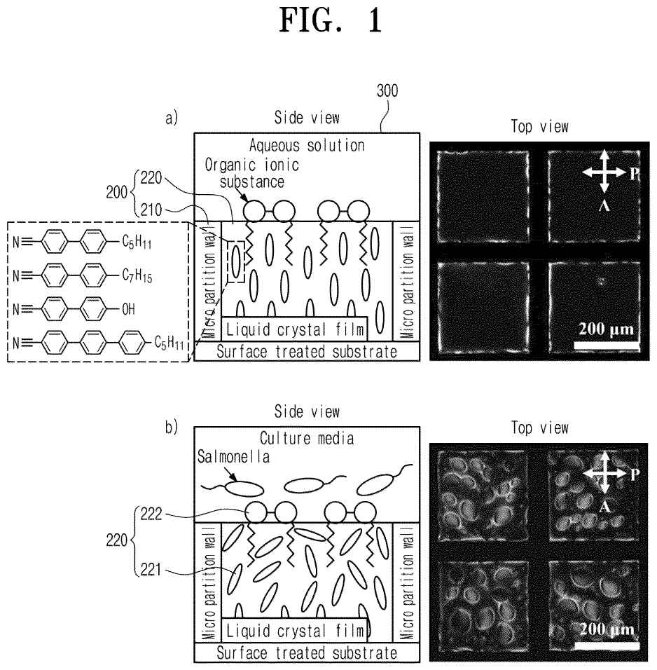

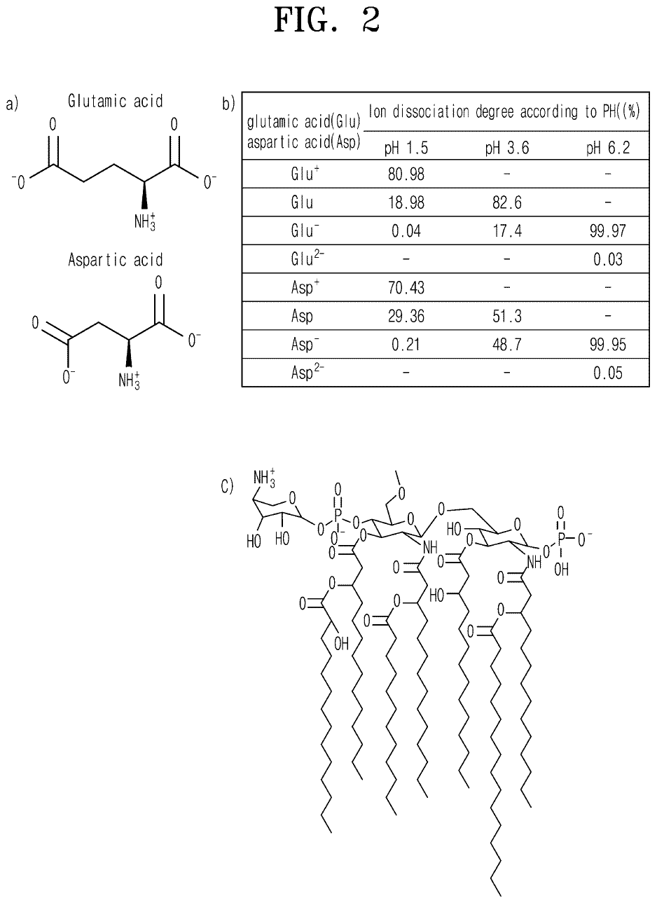

FIG. 1 is a view for schematically describing a food poisoning bacteria detection sensor according to an embodiment of the present disclosure, and FIG. 2 is a view for describing the food poisoning bacteria detection sensor according to an embodiment of the present disclosure in detail.

FIG. 1A is a view schematizing an appearance of the food poisoning bacteria detection sensor before use and showing optical characteristics thereof under a polarized optical microscope, and FIG. 1B is a view schematizing an appearance of the food poisoning bacteria detection sensor after use and showing optical characteristics thereof under the polarized optical microscope.

FIG. 2A is a view for describing charged side chain amino acid, FIG. 2B is a view for describing an ion dissociation degree according to pH of the charged side chain amino acid, and FIG. 2C is a view for describing lipopolysaccharide.

First, the food poisoning bacteria detection sensor according to an embodiment of the present disclosure includes a substrate part 100, a liquid crystal part 200 that is formed on the substrate part 100 and is composed of a micro partition wall 210 partitioning a unit pixel and a liquid crystal structure layer 220 disposed inside the partitioned unit pixel, and an accommodating part 300 that accommodates the substrate part 100 and the liquid crystal part 200 and includes an aqueous solution.

The substrate part 100 means a solid-phase substrate capable of supporting a liquid crystal, and any substrate to be usable for the application in the art may be used including an inorganic substance.

The inorganic substance includes quartz, silica, silver, gold, aluminum, copper, other silicon-including substances, metal oxide, and metal alloys, but it is not limited thereto.

According to a specific implementation of the present disclosure, the substrate part 100 of the present disclosure may be a glass substrate, and more specifically, the substrate part 100 may include a glass substrate coated with a coating material inducing liquid crystal molecules to be oriented perpendicular to a substrate-liquid crystal structure layer interface part.

Here, the coating material may be dimethyloctadecyl 3-(trimethoxysilyl)propyl ammonium chloride (DMOAP).

That is, in a preferred embodiment of the present disclosure, the substrate part 100 may be a glass substrate coated with dimethyloctadecyl 3-(trimethoxysilyl)propyl ammonium chloride (DMOAP).

The liquid crystal part 200 may be disposed to be partitioned into plural on the substrate part 100 through the micro partition wall 210 and include the liquid crystal structure layer 220.

In addition, a thickness of the liquid crystal part 200 may satisfy a range of 3 to 20 μm.

Here, the liquid crystal structure layer 220 may include a liquid crystal structure 221 that is composed of the liquid crystal molecules oriented perpendicular to the substrate-liquid crystal structure layer interface part and an aqueous solution-liquid crystal structure layer interface part, and an organic ionic substance 222 that is disposed at the aqueous solution-liquid crystal structure layer interface part.

The liquid crystal molecules of the liquid crystal structure 221 function to enable detection of food poisoning bacteria through an orientation change and may be used without particular limitation as long as they are substances that may be added to the liquid crystal of the sensor in general, but specifically may include any one substance selected from a group consisting of 4-n-octyl-4-cyano-biphenyl (8CB), 4-cyano-4′-pentylbiphenyl (5CB), and 4-pentyl-4′-cyanobiphenyl, and a mixture (E7) of 4-pentyl-4′-cyanobiphenyl, 4-heptyl-4′-cyanobiphenyl, 4-octyl-4′-cyanobiphenyl, and 4-pentyl-4′-cyanoterphenyl, and E7 may be used as the liquid crystal molecules in a preferred embodiment of the present disclosure.

In an embodiment of the present disclosure, an ‘orientation’ of the liquid crystal molecules means a result observed using a polarized optical microscope (POM) and a result observed from a photograph taken thereof, and may be divided into three types: perpendicular orientation, horizontal orientation, and tilting orientation. In addition, an ‘orientation change’ may mean that the orientation of the liquid crystal molecules changes from one of the three orientations to another, or that a directionality changes in one of the orientations.

In another embodiment of the present disclosure, an ‘orientation’ of liquid crystal molecules may be divided into four types: radial orientation, pre-radial orientation, axial orientation, and bipolar orientation, and an ‘orientation change’ may mean that the orientation of the liquid crystal molecules changes from one of the four orientations to another, or that a directionality changes in one of the orientations.

The organic ionic substance 222 may be fixedly disposed by being self-assembled at the aqueous solution-liquid crystal structure layer interface part and may induce the liquid crystal molecules to be oriented perpendicular to the aqueous solution-liquid crystal structure layer interface part.

The organic ionic substance 222 may include a head group, a carbon chain group, and an anion, and in a specific example, the head group may be any one substance selected from a sulfate group and an imidazolium group, a length of the carbon chain group may be 8 to 12, and the anion may be any one substance selected from Br⁻, BF₄⁻, and PF₆⁻.

In a preferred embodiment of the present disclosure, the organic ionic substance 222 may be any one substance of C₈—BI—PF₆, C₉—BI—PF₆, C₁₀—BI—PF₆, C₁₁—BI—PF₆, and C₁₂—BI—PF₆, but it is not limited thereto.

In addition, the organic ionic substance 222 may serve to precisely adjust a sensitivity of the food poisoning bacteria detection sensor of the present disclosure, and specifically, the length of the carbon chain group included in the organic ionic substance 222 may be selected according to the sensitivity of the sensor and target food poisoning bacteria.

Referring to FIGS. 1 and 2 again, in the food poisoning bacteria detection sensor according to an embodiment of the present disclosure, an optical appearance of the liquid crystal structure 221 may be changed according to an orientation change of the liquid crystal molecules, and this optical appearance change may enable immediate detection of an optical signal through the food poisoning bacteria detection sensor.

In the present disclosure, “optical appearance” means that when a liquid crystal between two polarizers positioned perpendicular to each other is observed with naked eye or with a suitable optical device, a bright-dark change is exhibited, and the liquid crystal changing from bright to dark or from dark to bright according to the optical appearance of the liquid crystal may be observed.

Specifically, in the food poisoning bacteria detection sensor according to an embodiment of the present disclosure, an orientation of the liquid crystal molecules of the liquid crystal structure 221 may be changed in a predetermined direction by at least one of food poisoning bacteria culture media and a food poisoning bacteria mixture in which food poisoning bacteria and the food poisoning bacteria culture media are mixed flowing into the accommodating part 300.

In an embodiment of the present disclosure, the orientation change of the liquid crystal molecules included in the liquid crystal structure 221 may be a change from a vertical orientation to a horizontal orientation.

Furthermore, the optical appearance change of the liquid crystal structure 221 may be induced by the orientation change of the liquid crystal molecules.

Here, the food poisoning bacteria culture media may include charged side chain amino including at least one substance selected from a group consisting of glutamic acid and aspartic acid. Furthermore, the food poisoning bacteria culture medium may include lysine.

The food poisoning bacteria culture media may further include various amino acids other than the charged side chain amino acid and the lysine, and as long as the amino acids are for culturing the food poisoning bacteria, they are not particularly limited.

The food poisoning bacteria are bacteria that include lipopolysaccharide (LPS) including a positively charged group and carbon chains as a major component of an outer membrane.

In a preferred embodiment of the present disclosure, the food poisoning bacteria may be infectious causative bacteria, and specifically, may be any one of pathogenic Escherichia coli, Vibrio parahaemolyticus, Salmonella, and Shigella.

More specifically, the food poisoning bacteria may be gram-negative bacteria that include lipopolysaccharide (LPS) including the positively charged group and the carbon chains as the major component of the outer membrane, and may most preferably be Salmonella.

The charged side chain amino acid induces the orientation change of the liquid crystal molecules by being adsorbed to the aqueous solution-liquid crystal structure layer interface part.

Specifically, when the charged side chain amino acid is flown into the aqueous solution, hydrogen of an OH group of the charged side chain amino acid is dissociated, that is, is ion-dissociated inside the aqueous solution to be negatively charged, thereby being adsorbed to the aqueous solution-liquid crystal structure layer interface part.

In addition, an adsorption amount of the interface part of the charged side chain amino acid may increase as a pH value of the aqueous solution increases.

Specifically, when the pH value of the aqueous solution changes, the ion dissociation degree of the charged side chain amino acid changes, and when the pH value increases, the dissociation of the OH group of the charged side chain amino acid is activated, thereby increasing an amount of negatively charged side chain amino acid.

On the other hand, when the pH value decreases, the amount of negatively charged side chain amino acid decreases as the dissociation of the OH group of the charged side chain amino acid is inactivated.

Furthermore, the liquid crystal molecules are oriented in a direction horizontal to the aqueous solution-liquid crystal structure layer interface part according to a change in an easy axis caused by adsorption of the charged side chain amino acid to the interface part.

Here, the change in the easy axis is induced as the liquid crystal molecules are oriented in a preferred direction by an interaction between the charged side chain amino acid and the liquid crystal, and is not by a formation of an isotropic phase of the liquid crystal.

In other words, an orientation change mechanism of the liquid crystal molecules may be based on the change in the easy axis caused by adsorption of the negatively charged side chain amino acid to the aqueous solution-liquid crystal structure layer interface part.

Meanwhile, the food poisoning bacteria may accelerate an adsorption speed of the charged side chain amino acid.

Specifically, the food poisoning bacteria include lipopolysaccharide (LPS) in an outer membrane thereof, and the lipopolysaccharide binds with the charged side chain amino acid to accelerate a speed at which the charged side chain amino acid is adsorbed to the aqueous solution-liquid crystal structure layer interface part.

Here, since the lipopolysaccharide includes a positively charged group, an electrostatic interaction between the positively charged group and the negatively charged side chain amino acid is increased and the lipopolysaccharide may bind with the charged side chain amino acid to accelerate the adsorption of the charged side chain amino acid to the aqueous solution-liquid crystal structure layer interface part. In addition, since the lipopolysaccharide includes the carbon chains, an interaction between the carbon chains and the liquid crystal molecules is increased and the adsorption of the charged side chain amino acid to the aqueous solution-liquid crystal structure layer interface part is accelerated, and furthermore, the horizontal orientation of the liquid crystal molecules may be maintained by preventing desorption of the charged side chain amino acid adsorbed to the interface part.

For example, when the food poisoning bacteria are Salmonella, the lipopolysaccharide (LPS) of the Salmonella has a 2-amino-2-deoxy sugar group that is the positively charged group, thereby may stably interact with the negatively charged side chain amino acid.

In addition, a concentration of the food poisoning bacteria may serve to precisely control the sensitivity of the food poisoning bacteria detection sensor of the present disclosure, and specifically, as the concentration of the food poisoning bacteria increases, the sensitivity of the sensor may increase, and the concentration of the food poisoning bacteria may be selected according to the sensitivity of the sensor.

More specifically, the concentration of the food poisoning bacteria may satisfy about 10¹ cfu (colony formation unit) to 10⁸ cfu per unit volume (1 ml) of the food poisoning bacteria culture media.

In other words, when the food poisoning bacteria mixture flows into the accommodating part, a speed at which the liquid crystal molecules are changed in an orientation from vertical to horizontal increases as a concentration of the flown food poisoning bacteria increases.

Accordingly, the optical appearance change of the liquid crystal structure 221 is accelerated according to the speed increase in the orientation change.

In addition, as described above, the organic ionic substance 222 may serve to precisely control the sensitivity of the food poisoning bacteria detection sensor of the present disclosure.

Specifically, as the length of the carbon chain group included in the organic ionic substance 222 decreases, the sensitivity of the sensor may increase, and the length of the carbon chain group inside the organic ionic substance 222 may be 8 to 12.

In other words, the speed at which the liquid crystal molecules are changed in the orientation from vertical to horizontal increases as the length of the carbon chain group included in the organic ionic substance 222 decreases, and the optical appearance change of the liquid crystal structure 221 is accelerated according to the speed increase in the orientation change.

In addition, the sensitivity of the food poisoning bacteria detection sensor of the present disclosure may be precisely controlled according to a shape of the liquid crystal structure 221.

In this regard, a food poisoning bacteria detection sensor according to another embodiment of the present disclosure will be described. In addition, the description will be focused on differential configuration according to another embodiment and mutual configuration will be omitted.

First, the food poisoning bacteria detection sensor according to another embodiment of the present disclosure includes a substrate part 100, a liquid crystal part 200 that is formed on the substrate part 100 and is composed of a micro partition wall 210 partitioning a unit pixel and a liquid crystal structure layer 220 disposed inside the partitioned unit pixel, and an accommodating part 300 that accommodates the substrate part 100 and the liquid crystal part 200 and includes an aqueous solution.

Here, the liquid crystal structure layer 220 according to another embodiment may include a droplet-shaped liquid crystal structure 221a that is composed of liquid crystal molecules oriented in radial configuration and has a topological defect in a center thereof, and an organic ionic substance 222 that is disposed at an aqueous solution-droplet-shaped liquid crystal structure interface part.

In the food poisoning bacteria detection sensor according to another embodiment of the present disclosure, an optical appearance of the droplet-shaped liquid crystal structure 221a may be changed according to the orientation change of the liquid crystal molecules, and this change in the optical appearance may enable immediate detection of an optical signal through the food poisoning bacteria detection sensor.

In another embodiment of the present disclosure, an orientation change of the liquid crystal molecules included in the droplet-shaped liquid crystal structure 221a may be a change from a radial orientation to a bipolar orientation.

Specifically, in the food poisoning bacteria detection sensor according to another embodiment of the present disclosure, the orientation of the liquid crystal molecules included in the droplet-shaped liquid crystal structure 221a may be changed from the radial orientation to the bipolar orientation by any one of food poisoning bacteria culture media and a food poisoning bacteria mixture in which food poisoning bacteria and the food poisoning bacteria culture media are mixed flowing into the accommodating part.

Here, the food poisoning bacteria culture media may include charged side chain amino acid including at least one substance selected from a group consisting of glutamic acid and aspartic acid. Furthermore, the food poisoning bacteria culture medium may include lysine.

The charged side chain amino acid induces the orientation change of the liquid crystal molecules from the radial orientation to the bipolar orientation by being adsorbed to the aqueous solution-droplet-shaped liquid crystal structure interface part.

Specifically, when the charged side chain amino acid is flown into the aqueous solution, hydrogen of an OH group of the charged side chain amino acid is dissociated, that is, is ion-dissociated inside the aqueous solution to be negatively charged, thereby being adsorbed to the aqueous solution-droplet shaped liquid crystal structure interface part.

Furthermore, a position of the topological defect of the droplet-shaped liquid crystal structure 221a is changed according to the orientation change of the liquid crystal molecules of the aqueous solution-liquid crystal structure layer interface part caused by the adsorption of the charged side chain amino acid to the interface part, thereby having the bipolar orientation.

Like an embodiment of the present disclosure, in a food poisoning bacteria detection sensor according to another embodiment of the present disclosure, a speed at which liquid crystal molecules included in the droplet-shaped liquid crystal structure are changed in the orientation increases as a concentration of the flown food poisoning bacteria increases, the speed at which the liquid crystal molecules are changed in the orientation increases as a length of a carbon chain group included in the organic ionic substance decreases, and the optical appearance change of the droplet-shaped liquid crystal structure may be accelerated according to the speed increase in the orientation change.

### Method of Manufacturing Food Poisoning Bacteria Detection Sensor

A method of manufacturing a food poisoning bacteria detection sensor according to an embodiment of the present disclosure may include: a first step (S100) of preparing a substrate part; a second step (S200) of forming a liquid crystal part that is composed of a micro partition wall partitioning a unit pixel and a liquid crystal structure layer disposed inside the partitioned unit pixel on the substrate part; and a third step (S300) of disposing the substrate part in an accommodating part that accommodates the liquid crystal part and the substrate part and includes an aqueous solution.

The first step is preparing a solid substrate capable of supporting a liquid crystal, and may include coating a coating material on an inorganic substance including glass, quartz, silica, silver, gold, aluminum, copper, other silicon-containing substances, metal oxide, and metal alloys.

In a preferred embodiment of the present disclosure, the first step may be preparing a glass substrate coated with dimethyloctadecyl 3-(trimethoxysilyl)propyl ammonium chloride (DMOAP).

The second step is forming the liquid crystal part disposed to be partitioned into the unit pixel on the substrate part.

Specifically, in the second step, a partition wall with a height of 3 to 20□ is disposed on the prepared substrate, and then a liquid crystal mixture is applied inside the partition wall. In this case, the liquid crystal mixture may be obtained by mixing a liquid crystal and an organic ionic substance and then stirring the same using an ultrasonic stirrer for a predetermined time.

The organic ionic substance may be any one substance of C₈—BI—PF₆, C₉—BI—PF₆, C₁₀—BI—PF₆, C₁₁—BI—PF₆, and C₁₂—BI—PF₆, but it is not limited thereto.

Here, since surface energy in the aqueous solution-liquid crystal structure layer interface part may vary depending on a carbon chain group included in the organic ionic substance, concentrations of C₈—BI—PF₆, C₉—BI—PF₆, and C₁₀—BI—PF₆ are set to satisfy a range of 0.8 to 1.2 mM, and concentrations of C₁₁—BI—PF₆, and C₁₂—BI—PF₆ are set to satisfy a range of 0.1 to 0.3 mM.

The third step is disposing the substrate part on which the liquid crystal part is formed in the accommodating part, and the substrate part is disposed in the accommodating part including an aqueous solution to manufacture the food poisoning bacteria detection sensor.

### Example 1: Manufacturing of Food Poisoning Bacteria Detection Sensor 1

S100: A dimethyloctadecyl 3-(trimethoxysilyl)propyl ammonium chloride (DMOAP) coated glass substrate was prepared by coating dimethyloctadecyl 3-(trimethoxysilyl)propyl ammonium chloride (DMOAP) on a glass substrate.

S200: After disposing a micro partition wall with a height of 18□ on the DMOAP coated glass substrate, a liquid crystal mixture was applied inside the partition wall.

Here, the liquid crystal mixture was obtained by mixing a liquid crystal and an organic ionic substance and then stirring the same using an ultrasonic stirrer for 15 minutes.

In this case, E7 was used as the liquid crystal, and the organic ionic substance may be one of C₈—BI—PF₆, C₁₀—BI—PF₆, and C₁₂—BI—PF₆. In addition, in the organic ionic substance that is mixed with the liquid crystal, concentrations of C₈—BI—PF₆, and C₁₀—BI—PF₆ were set to satisfy a range of 1 mM, and a concentration of C₁₂—BI—PF₆ was set to satisfy a range of 0.2 mM.

S300: The glass substrate on which the liquid crystal mixture is coated was disposed in an accommodating part including an aqueous solution with pH values of 1.5, pH 3.6, and pH 6.2 to manufacture a food poisoning bacteria detection sensor.

In Example 1, liquid crystal molecules included in the liquid crystal mixture have a state of being oriented perpendicular to a substrate-liquid crystal structure layer interface part and an aqueous solution-liquid crystal structure layer interface part.

A food poisoning bacteria detection sensor including the micro partition wall with the height of 18□, the organic ionic substance of C₁₂—BI—PF₆, and the aqueous solution with the pH value of 6.2 is sample A of Example 1, a food poisoning bacteria detection sensor including the micro partition wall with the height of 18□, the organic ionic substance of C₁₂—BI—PF₆, and the aqueous solution with the pH value of 3.6 is sample B of Example 1, and a food poisoning bacteria detection sensor including the micro partition wall with the height of 18□, the organic ionic substance of C₁₂—BI—PF₆, and the aqueous solution with the pH value of 1.5 is sample C of Example 1,

In addition, a food poisoning bacteria detection sensor including the micro partition wall with the height of 18□, the organic ionic substance of C₁₀—BI—PF₆, and the aqueous solution with the pH value of 6.2 is sample D of Example 1, and a food poisoning bacteria detection sensor including the micro partition wall with the height of 18□, the organic ionic substance of C₈—BI—PF₆, and the aqueous solution with the pH value of 6.2 is sample E of Example 1.

### Example 2: Manufacturing of Food Poisoning Bacteria Detection Sensor Based on Droplet-Shaped Liquid Crystal Structure

S100: A glass substrate was prepared.

S200: After disposing a micro partition wall with a height of 18□ on the glass substrate, a liquid crystal mixture was applied inside the partition wall.

Here, the liquid crystal mixture was obtained by mixing a liquid crystal and an organic ionic substance and then stirring the same using an ultrasonic stirrer for 15 minutes.

In this case, E7 was used as the liquid crystal, and the organic ionic substance may be one of C₈—BI—PF₆, C₁₀—BI—PF₆, and C₁₂—BI—PF₆. In addition, in the organic ionic substance that is mixed with the liquid crystal, concentrations of C₈—BI—PF₆, and C₁₀—BI—PF₆ were set to satisfy a range of 1 mM, and a concentration of C₁₂—BI—PF₆ was set to satisfy a range of 0.2 mM.

S300: The glass substrate on which the liquid crystal mixture is coated was disposed in an accommodating part including an aqueous solution with a pH value of pH 6.2 to manufacture a food poisoning bacteria detection sensor.

In Example 2, liquid crystal molecules included in the liquid crystal mixture have a state of being oriented in radial configuration.

A food poisoning bacteria detection sensor including the micro partition wall with the height of 18□, the organic ionic substance of C₁₂—BI—PF₆, and the aqueous solution with the pH value of 6.2 is sample A of Example 2, a food poisoning bacteria detection sensor including the micro partition wall with the height of 18□, the organic ionic substance of C₁₀—BI—PF₆, and the aqueous solution with the pH value of 6.2 is sample B of Example 2, and a food poisoning bacteria detection sensor including the micro partition wall with the height of 18□, the organic ionic substance of C₈—BI—PF₆, and the aqueous solution with the pH value of 6.2 is sample C of Example 2,

### Comparative Example 1: Manufacturing of Food Poisoning Bacteria Detection Sensor 3

S100: A dimethyloctadecyl 3-(trimethoxysilyl)propyl ammonium chloride (DMOAP) coated glass substrate was prepared by coating dimethyloctadecyl 3-(trimethoxysilyl)propyl ammonium chloride (DMOAP) on a glass substrate.

S200: After disposing a micro partition wall with a height of 3.6□ on the DMOAP coated glass substrate, a liquid crystal mixture was applied inside the partition wall.

Here, the liquid crystal mixture may be a liquid crystal.

In this case, E7 was used as the liquid crystal.

S300: The glass substrate on which the liquid crystal mixture is coated was disposed in an accommodating part including an aqueous solution with a pH value of pH 6.2 to manufacture a food poisoning bacteria detection sensor.

Here, liquid crystal molecules included in the liquid crystal mixture have a state of being oriented perpendicular to a substrate-liquid crystal structure layer interface part and an aqueous solution-liquid crystal structure layer interface part.

### Comparative Example 2: Manufacturing of Food Poisoning Bacteria Detection Sensor 4

S100: A polyimide coated glass substrate was prepared by coating polyimide on a glass substrate.

S200: After disposing a micro partition wall with a height of 3.6□ on a DMOAP coated glass substrate, a liquid crystal mixture was applied inside the partition wall.

Here, the liquid crystal mixture may be a liquid crystal.

In this case, E7 was used as the liquid crystal.

S300: The glass substrate on which the liquid crystal mixture is coated was disposed in an accommodating part including an aqueous solution with a pH value of pH 6.2 to manufacture a food poisoning bacteria detection sensor.

In addition, by utilizing a general rubbing method, liquid crystal molecules included in the liquid crystal mixture were configured to be oriented horizontally to a substrate-liquid crystal structure layer interface part and an aqueous solution-liquid crystal structure layer interface part.

### Experimental Example 1: Evaluation of Optical Signal Detection

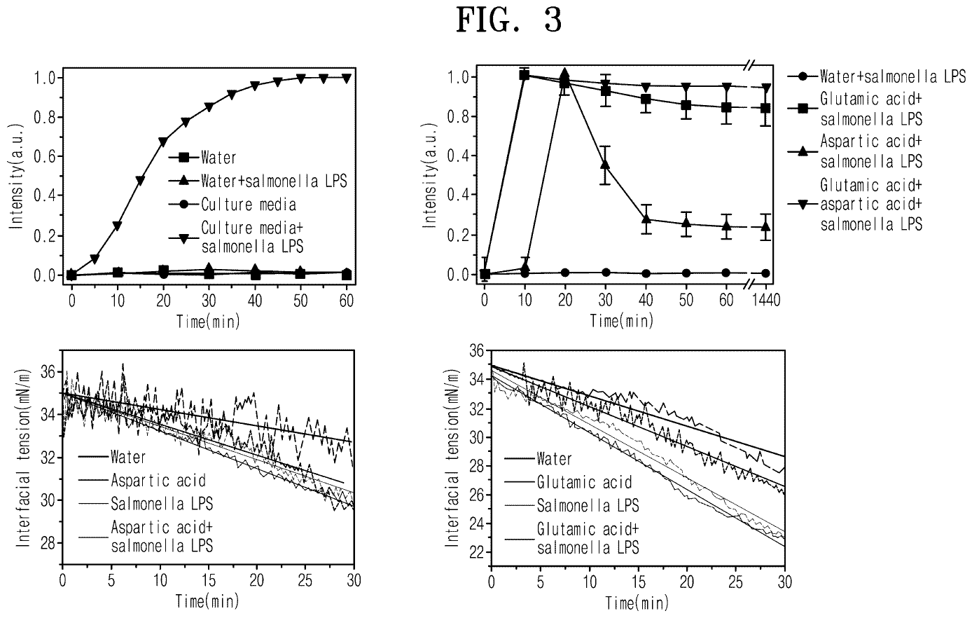

Sample A according to Example 1 was prepared, and at least one substance of water, Salmonella lipopolysaccharide (LPS), Salmonella culture media, glutamic acid, and aspartic acid was added to the prepared sample A according to Example 1 to evaluate optical signal detection and interfacial tension change, and an evaluation result is shown in FIG. 3.

FIG. 3 is a view showing an optical signal detection result of a food poisoning bacteria detection sensor according to the present disclosure.

FIGS. 3A and 3B are graphs of an optical signal change of sample A according to Example 1, and FIGS. 3C and 3D are graphs of an interfacial tension change of sample A according to Example 1.

As shown in FIG. 3A, when a flown substance flowing into a sensor is water, water and Salmonella lipopolysaccharide (LPS), Salmonella culture media, and Salmonella culture media and Salmonella lipopolysaccharide (LPS), it was confirmed that when the Salmonella lipopolysaccharide (LPS) was flown together with the culture media, LPS bound with glutamic acid and aspartic acid in the culture media to accelerate adsorption of the glutamic acid and the aspartic acid to the aqueous solution-liquid crystal interface, thereby accelerating an orientation of liquid crystal molecules in a direction horizontal to the aqueous solution-liquid crystal interface.

As shown in FIG. 3B, when a flown substance flowing into the sensor is water and Salmonella lipopolysaccharide (LPS), glutamic acid and Salmonella lipopolysaccharide (LPS), aspartic acid and Salmonella lipopolysaccharide (LPS), and glutamic acid and aspartic acid and Salmonella lipopolysaccharide (LPS), it was confirmed that when the Salmonella lipopolysaccharide (LPS) was flown together with at least one substance selected from the glutamic acid and the aspartic acid, a generation of the optical signal was accelerated according to an interaction between the LPS, charged side chain amino acids (glutamic acid, aspartic acid), and the liquid crystal molecules.

As shown in FIG. 3C, when a flown substance flowing into the sensor is water, aspartic acid, Salmonella lipopolysaccharide (LPS), and aspartic acid and Salmonella lipopolysaccharide (LPS), it was confirmed that a decrease in the interfacial tension was accelerated when the LPS and the aspartic acid were flown together.

As shown in FIG. 3D, when a flown substance flowing into the sensor is water, glutamic acid, Salmonella lipopolysaccharide (LPS), and glutamic acid and Salmonella lipopolysaccharide (LPS), it was confirmed that the decrease in the interfacial tension was accelerated when the LPS and the glutamic acid were flown together.

### Experimental Example 2: Evaluation of Optical Signal Detection According to pH

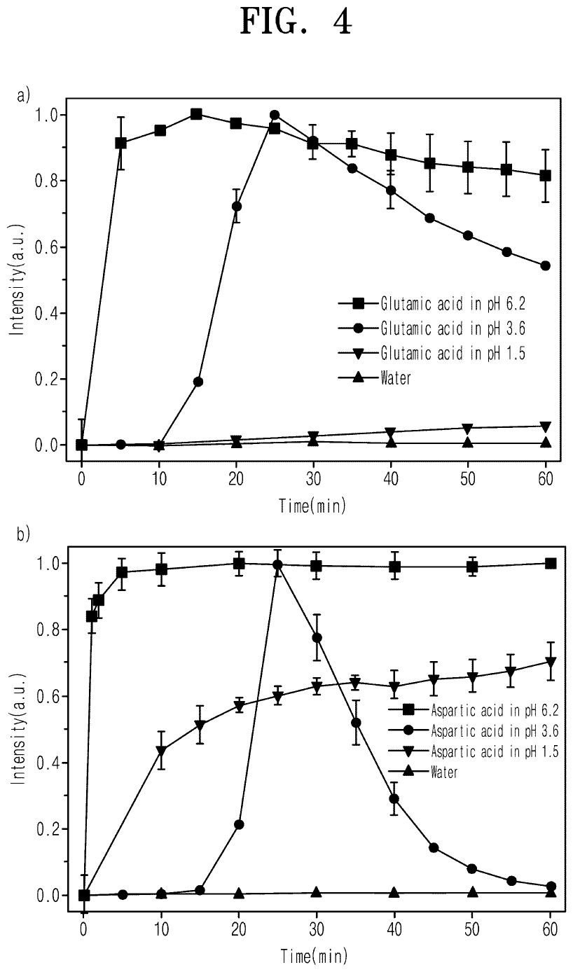

Samples, A, B, and C according to Example 1 were prepared, and either one substance of glutamic acid and aspartic acid was added to the prepared samples A, B, and C to evaluate optical signal detection, and an evaluation result is shown in FIG. 4.

FIG. 4A is a graph of an optical signal change according to a flow of glutamic acid into samples A, B, and C according to Example 1, and FIG. 4B is a graph of an optical signal change according to a flow of aspartic acid into samples A, B, and C according to Example 1.

As shown in FIG. 4, it was confirmed that the glutamic acid or the aspartic acid induces an orientation change of liquid crystal molecules, and as a pH value of an aqueous solution in a sensor increases, a ratio of anionic glutamic acid and anionic aspartic acid increases, thereby accelerating a generation of the optical signal by increasing an adsorption amount of an interface part of the glutamic acid and the aspartic acid.

### Experimental Example 3: Evaluation of Optical Retardation

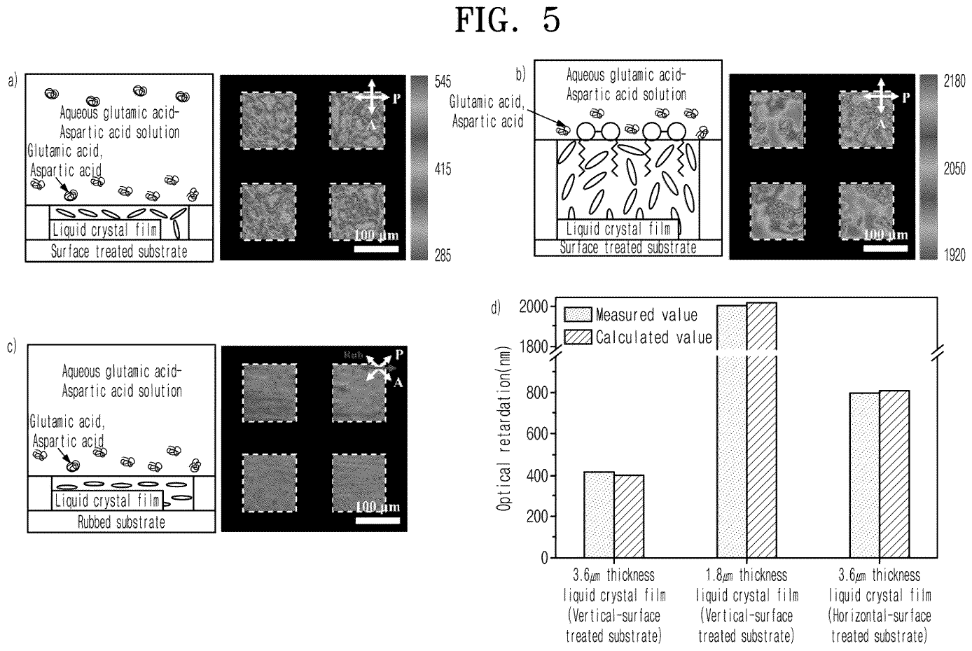

Sample A according to Example 1 and Comparative Example 1 and Comparative Example 2 were prepared, and glutamic acid and aspartic acid were added to the prepared sample A, Comparative Example 1 and Comparative Example 2 to measure a magnitude of optical retardation of a liquid crystal. Specifically, a variable retarder-coupled polarization microscope (POLSCOPE) was used to measure the magnitude of the optical retardation of the prepared sample, and a measurement result is shown in FIG. 5.

FIG. 5A is a view schematizing Comparative Example 1, FIG. 5B is a view schematizing sample A according to Example 1, FIG. 5C is a view schematizing Comparative Example 2, and FIG. 5D is a graph comparing a measured value and a calculated value of the optical retardation.

As shown in FIG. 5, it was confirmed that in sample A according to Comparative Example 1 and Example 1, liquid crystal molecules were oriented to a horizontal direction from a direction perpendicular to an aqueous solution-liquid crystal interface as Salmonella culture media was flown into sample A. In addition, as a result of comparing the measured value and the calculated value of the optical retardation, it was confirmed that two values were almost identical and the value of the optical retardation was not 0, and thus the liquid crystal molecules were not an isotropic phase.

In addition, in Comparative Example 2, it was confirmed that as the Salmonella culture media was flown, an orientation change of the liquid crystal molecules in the direction horizontal to the aqueous solution-liquid crystal interface did not occur. In addition, as a result of comparing the measured value and the calculated value of the optical retardation, it was confirmed that the two values were almost identical, and the value of the optical retardation was not 0, and thus the liquid crystal molecules were not the isotropic phase.

That is, through optical retardation measurement, it was confirmed that the orientation change of the liquid crystal caused by the Salmonella culture media was not due to a formation of the isotropic phase of the liquid crystal but was due to a change in the easy axis of the liquid crystal.

### Experimental Example 4: Evaluation of Optical Signal Detection According to Salmonella Concentration

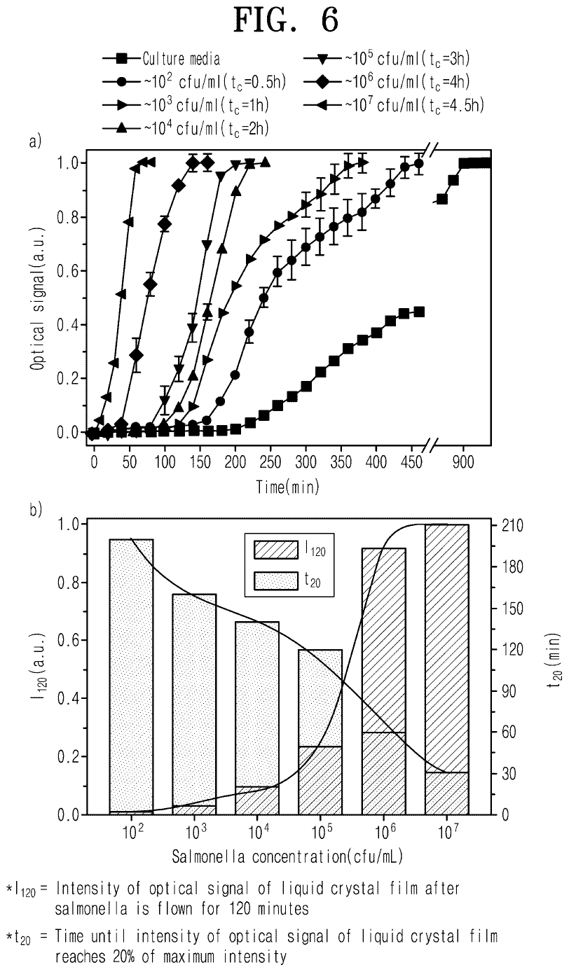

Sample A according to Example 1 was prepared, Salmonella culture media and a mixture of the Salmonella culture media and Salmonella were added to the prepared sample A according to Example 1 to evaluate optical signal detection, and an evaluation result is shown in FIG. 6.

A concentration of the Salmonella was set to satisfy about 10² cfu (colony formation unit) to 10⁷ cfu per unit volume (1 ml) of the Salmonella culture media.

FIG. 6A is a graph of an optical signal change over time of sample A, and FIG. 6B is a view showing an intensity of an optical signal after the Salmonella is flown into sample A for 120 minutes according to the concentration of the Salmonella and a time until the intensity of the optical signal reaches 20% of the maximum intensity.

As shown in FIG. 6, it was confirmed that as the concentration of the Salmonella flowing into a sensor increases, a horizontal orientation of liquid crystal molecules to the aqueous solution-liquid crystal interface is accelerated, thereby accelerating a generation speed of the optical signal.

In addition, it was confirmed that as the concentration of the Salmonella increases, I₁₂₀ (an intensity of the optical signal after the Salmonella is flown for 120 minutes) increases and t₂₀ (a time until the intensity of the optical signal reaches 20% of the maximum intensity) decreases.

Accordingly, it was confirmed that it is possible to measure not only a presence of the Salmonella but also a quantitative concentration of the Salmonella using the intensity of the optical signal.

### Experimental Example 5: Evaluation of Optical Signal Detection According to Length of Carbon Chain Group of Organic Ionic Substance

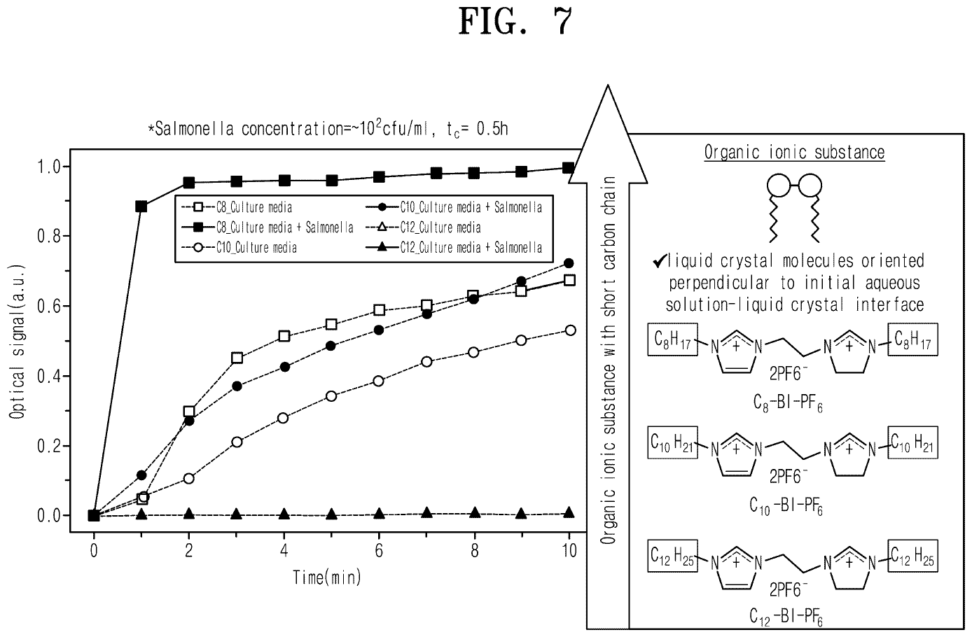

Sample A, sample D, and sample E according to Example 1 were prepared, Salmonella culture media and a mixture of the Salmonella culture media and the Salmonella were added to the prepared sample A, sample D, and sample E according to Example 1 to evaluate optical signal detection, and an evaluation result is shown in FIG. 7.

A concentration of the Salmonella was set to satisfy about 10² cfu (colony formation unit) per unit volume (1 ml) of the Salmonella culture media.

FIG. 7 is a graph of an optical signal change according to a length of a carbon chain group included in an organic ionic substance.

As shown in FIG. 7, it was confirmed that as the length of the carbon chain group included in the organic ionic substance decreases, a generation speed of an optical signal of a sensor is accelerated.

### Experimental Example 6: Evaluation of Optical Signal Detection of Food Poisoning Bacteria Detection Sensor Based on Droplet-Shaped Liquid Crystal Structure

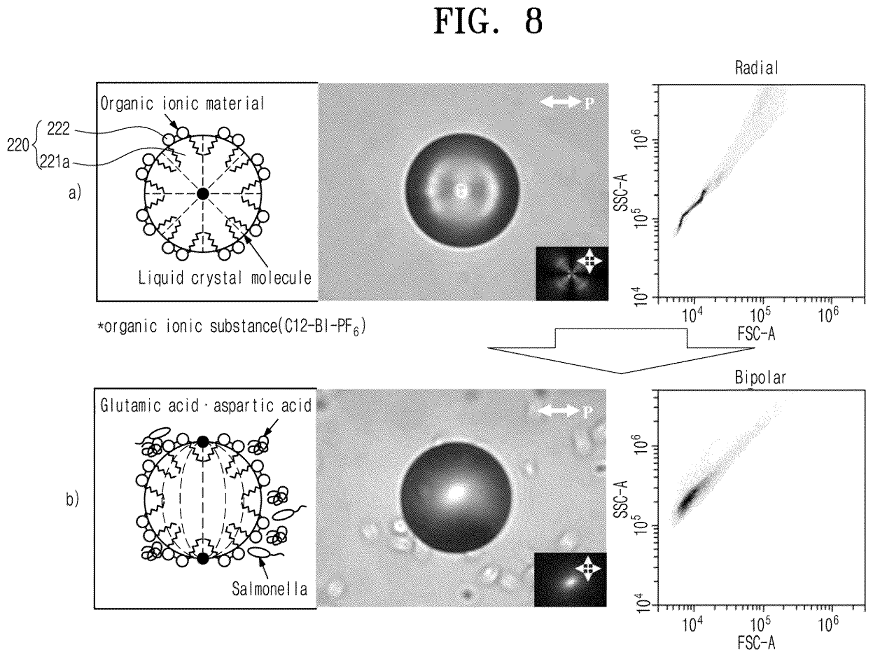

Sample A according to Example 2 was prepared, a mixture of Salmonella culture media and Salmonella was added to the prepared sample A to evaluate optical signal detection, and an evaluation result is shown in FIG. 8.

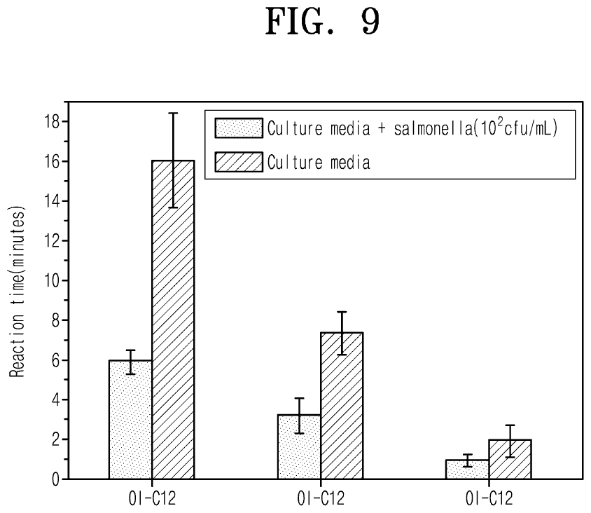

Sample A, sample B, and sample C according to Example 2 were prepared, the Salmonella culture media and a mixture of the Salmonella culture media and the Salmonella were added to the prepared sample A, sample B, and sample C to evaluate a reaction time, and an evaluation result is shown in FIG. 9.

A concentration of the Salmonella was set to satisfy about 10² cfu (colony formation unit) per unit volume (1 ml) of the Salmonella culture media.

FIG. 8A is a view schematizing an appearance of sample A according to Example 2 before the mixture of the Salmonella culture media and the Salmonella is flown and showing optical characteristics thereof under an optical microscope, and FIG. 8B is a view schematizing an appearance of sample A according to Example 2 after the mixture of the Salmonella culture media and the Salmonella is flown and showing optical characteristics thereof under the optical microscope.

FIG. 9 is a graph of a reaction time change according to a length of a carbon chain included in an organic ionic substance.

As shown in FIG. 8, an orientation change of liquid crystal molecules included in a droplet-shaped liquid crystal structure may be a change from a radial orientation to a bipolar orientation, and when the mixture of the Salmonella culture media and the Salmonella flows into sample A according to Example 2, as at least one substance of glutamic acid and aspartic acid is adsorbed to an interface part, a position of a topological defect of the liquid crystal molecules included in the droplet-shaped liquid crystal structure is changed according to the orientation change of the liquid crystal molecules of an aqueous solution-liquid crystal structure layer interface part, thereby changing from the radial orientation to the bipolar orientation.

Accordingly, it was confirmed that a light scattering pattern analysis was possible by the orientation change of the liquid crystal molecules included in the droplet-shaped liquid crystal structure.

As shown in FIG. 9, it was confirmed that a generation speed of an optical signal of the samples according to Example 2 increased when the Salmonella culture media and the Salmonella were flown together, and furthermore, it was confirmed that as the length of the carbon chain group included in the organic ionic substance decreases, the generation speed of an optical signal of a sensor is accelerated.

### Experimental Example 7: Evaluation of Salmonella Selectivity

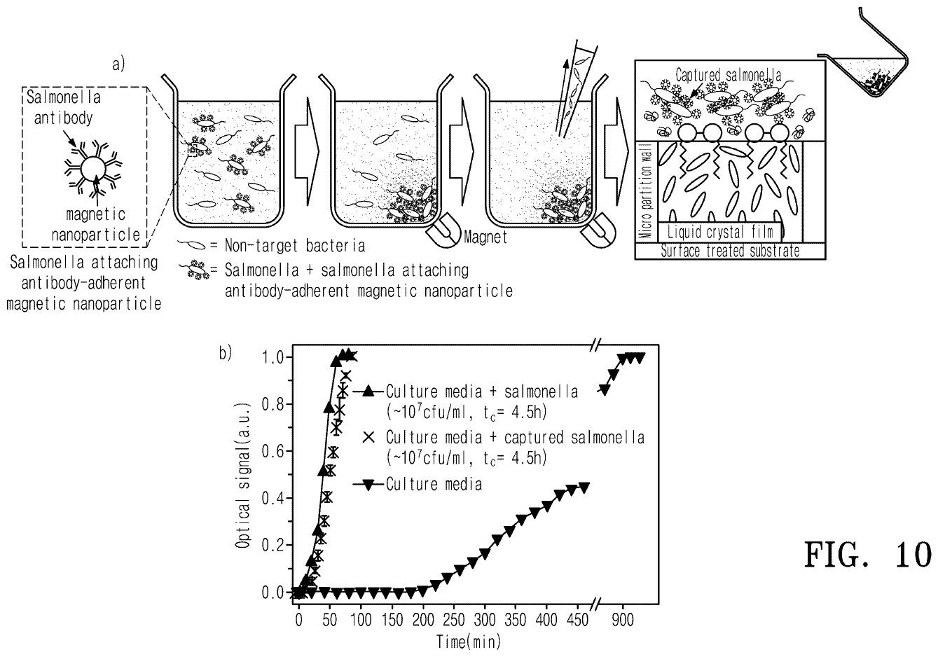

Sample A according to Example 1 was prepared, culture media, a capture mixture including Salmonella culture media and captured Salmonella, and a mixture of the Salmonella culture media and the Salmonella were added to the prepared sample A according to Example 1 to evaluate optical signal detection, and an evaluation result is shown in FIG. 10.

Specifically, the capture mixture may include captured Salmonella obtained by mixing the Salmonella that is a target material with a non-target material, attaching antibody-adherent magnetic nanoparticles to the Salmonella, and then separately capturing only the Salmonella to which the magnetic nanoparticles are attached.

The captured Salmonella was captured from the Salmonella with about 10⁷ cfu (colony formation unit) per unit volume (1 ml) of the Salmonella culture media, and a capture ratio was set to satisfy about 83%.

A concentration of the Salmonella was set to satisfy about 10⁷ cfu (colony formation unit) per unit volume (1 ml) of the Salmonella culture media.

FIG. 10A is a view for describing the captured Salmonella, and FIG. 10B is a graph of an optical signal change of sample A according to Example 1 when the captured Salmonella or general Salmonella is flown.

As shown in FIG. 10, it was confirmed that there was almost no difference in speed when comparing a generation speed of the optical signal when the capture mixture including the Salmonella culture media and the captured Salmonella was flown into a sensor with a generation speed of the optical signal when the mixture including the Salmonella culture media and the general Salmonella was flown into the sensor.

That is, it was confirmed that a target Salmonella may be selectively detected within a short period of time by concentrating the Salmonella using a magnet or the like without a Salmonella enrichment process.

### Experimental Example 8: Simulation Evaluation of Interaction of Salmonella and Charged Side Chain Amino Acid

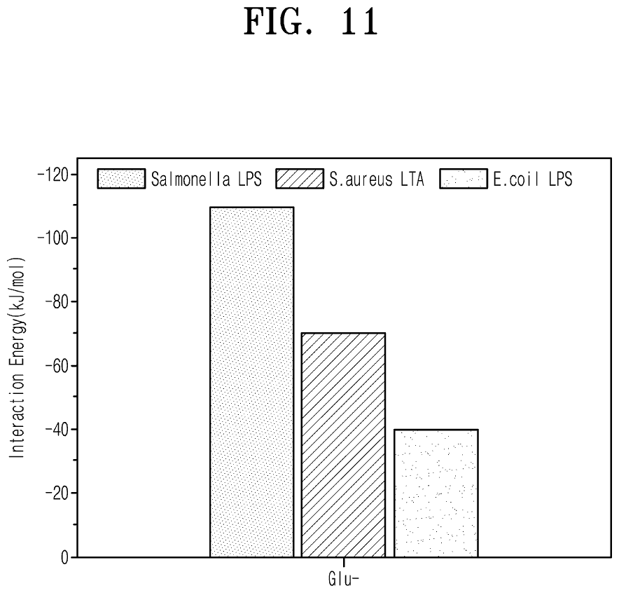

Interaction energy between at least one substance of Salmonella that includes lipopolysaccharide (LPS) including a positively charged group and carbon chains as a major component of an outer membrane, Staphylococcus aureus that includes lipoteichoic acid (LTA) including a neutral group and carbon chains as a major component of cell wall, and Escherichia coli that includes lipopolysaccharide (LPS) including a negatively charged group and carbon chains as a major component of an outer membrane, and glutamic acid (Glu) that is a charged side chain amino acid is evaluated by simulation through density functional theory (DFT) calculation, and an evaluation result is shown in FIG. 11.

FIG. 11 is an evaluation result of interaction energy between bacteria and charged side chain amino acid.

As shown in FIG. 11, it was confirmed that lipopolysaccharide of Salmonella (Salmonella LPS) had the lowest interaction energy with negatively charged glutamic acid as it included the positively charged group, lipopolysaccharide of E. coli (E. coli LPS) had the highest interaction energy with the negatively charged glutamic acid as it included the negatively charged group, and lipoteichoic acid of Staphylococcus aureus (S. aureus LTA) had an interaction energy with the negatively charged glutamic acid higher than lipopolysaccharide-glutamic acid interaction energy of the Salmonella and lower than lipopolysaccharide-glutamic acid interaction energy of E. coli as it included a neutral group.

Accordingly, it was confirmed through simulation that the lipopolysaccharide of the Salmonella may stably interact with glutamic acid (Glu) that is a negatively charged side chain amino acid as it includes the positively charged group (2-amino-2-deoxy sugar group) and that the lipopolysaccharide of the Salmonella may bind with the charged side chain amino acid to accelerate a speed at which the charged side chain amino acid is adsorbed to an aqueous solution-liquid crystal structure layer interface part.

Although the food poisoning bacteria detection sensor according to the embodiments of the present disclosure have been described as specific embodiments, it is merely an example, and the present disclosure is not limited thereto and it should be interpreted to have the broadest scope in accordance with the basic idea disclosed in this specification. Those skilled in the art may combine and substitute the disclosed embodiments to implement embodiments not specified, but this also does not depart from the scope of the present disclosure. In addition, those skilled in the art may easily change or modify the disclosed embodiments based on this specification, and it is clear that such changes or modifications also fall within the scope of the present disclosure.

What is claimed is:

1. A food poisoning bacteria detection sensor comprising:
a substrate part;
a liquid crystal part that is formed on the substrate part and is composed of a micro partition wall partitioning a unit pixel and a liquid crystal structure layer disposed inside the partitioned unit pixel; and
an accommodating part that accommodates the substrate part and the liquid crystal part and includes an aqueous solution.

2. The food poisoning bacteria detection sensor of claim 1,
wherein the liquid crystal structure layer includes
a liquid crystal structure that is composed of liquid crystal molecules oriented perpendicular to a substrate-liquid crystal structure layer interface part and an aqueous solution-liquid crystal structure layer interface part, and an organic ionic substance that is disposed at the aqueous solution-liquid crystal structure layer interface part.

3. The food poisoning bacteria detection sensor of claim 2,
wherein the substrate part includes
a glass substrate on which a coating material inducing the liquid crystal molecules to be oriented perpendicular to the substrate-liquid crystal structure layer interface part is coated, and
the coating material is dimethyloctadecyl 3-(trimethoxysilyl)propyl ammonium chloride (DMOAP).

4. The food poisoning bacteria detection sensor of claim 3,
wherein the liquid crystal molecules are changed in an orientation in a predetermined direction by at least one of food poisoning bacteria culture media flowing into the accommodating part and a food poisoning bacteria mixture in which food poisoning bacteria and the food poisoning bacteria culture media are mixed, and
an optical appearance change of the liquid crystal structure is induced by the orientation change of the liquid crystal molecules.

5. The food poisoning bacteria detection sensor of claim 4,
wherein the food poisoning bacteria culture media includes
charged side chain amino acid including at least one substance selected from a group consisting of glutamic acid and aspartic acid, and
the food poisoning bacteria are bacteria that include lipopolysaccharide including a positively charged group and carbon chains as a major component of an outer membrane.

6. The food poisoning bacteria detection sensor of claim 5,
wherein the charged side chain amino acid induces the orientation change of the liquid crystal molecules by being adsorbed to the aqueous solution-liquid crystal structure layer interface part, and
the liquid crystal molecules are oriented in a direction horizontal to the aqueous solution-liquid crystal structure layer interface part according to a change in an easy axis caused by adsorption of the side chain amino acid to the interface part.

7. The food poisoning bacteria detection sensor of claim 6,
wherein an adsorption amount of the interface part of the charged side chain amino acid increases as a pH value of the aqueous solution increases, and
the lipopolysaccharide binds with the charged side chain amino acid to accelerate a speed at which the side chain amino acid is adsorbed to the aqueous solution-liquid crystal structure layer interface part.

8. The food poisoning bacteria detection sensor of claim 7,
wherein the organic ionic substance is fixedly disposed by being self-assembled at the aqueous solution-liquid crystal structure layer interface part and induces the liquid crystal molecules to be oriented perpendicular to the aqueous solution-liquid crystal structure layer interface part.

9. The food poisoning bacteria detection sensor of claim 8,
wherein the organic ionic substance includes
a head group, a carbon chain group, and an anion,
the head group is any one substance selected from a sulfate group and an imidazolium group,
a length of the carbon chain group is 8 to 12, and
the anion is any one substance selected from Br⁻, BF₄⁻, and PF₆⁻.

10. The food poisoning bacteria detection sensor of claim 9,
wherein, when the food poisoning bacteria mixture flows into the accommodating part, a speed at which the liquid crystal molecules are changed in an orientation from vertical to horizontal increases as a concentration of the flown food poisoning bacteria increases, and
the optical appearance change of the liquid crystal structure is accelerated according to the speed increase in the orientation change.

11. The food poisoning bacteria detection sensor of claim 10,
wherein the speed at which the liquid crystal molecules are changed in the orientation from vertical to horizontal increases as a length of the carbon chain group included in the organic ionic substance decreases, and
the optical appearance change of the liquid crystal structure is accelerated according to the speed increase in the orientation change.

12. The food poisoning bacteria detection sensor of claim 1,
wherein the liquid crystal structure layer includes
a droplet-shaped liquid crystal structure that is composed of liquid crystal molecules oriented in radial configuration and having a topological defect in a center thereof, and an organic ionic substance disposed at an aqueous solution-droplet-shaped liquid crystal structure interface part.

13. A method of manufacturing a food poisoning bacteria detection sensor comprising:
a first step of preparing a substrate part;
a second step of forming a liquid crystal part that is composed of a micro partition wall partitioning a unit pixel and a liquid crystal structure layer disposed inside the partitioned unit pixel on the substrate part; and
a third step of disposing the substrate part in an accommodating part that accommodates the liquid crystal part and the substrate part and includes an aqueous solution.

14. A method of detecting food poisoning bacteria, the method comprising:
bringing a sample to be in contact with the substrate part of the food poisoning bacteria detection sensor of claim 1; and
detecting whether food poisoning bacteria is present in the sample.
